(19) United States
(12) Patent Application Publication
(10) Pub. No.: US 2019/0008680 A1
(43) Pub. Date: Jan. 10, 2019

(54) PORT FEATURES FOR ULTRASONIC VITRECTOMY TIP

(71) Applicant: Novartis AG, Basel (CH)

(72) Inventors: Mauricio Jochinsen, Fountain Valley, CA (US); Omeed Paydar, Irvine, CA (US)

(21) Appl. No.: 15/954,075

(22) Filed: Apr. 16, 2018

Related U.S. Application Data
(60) Provisional application No. 62/529,241, filed on Jul. 6, 2017.

(51) Int. Cl. A61F 9/007

(52) U.S. Cl. CPC A61F 9/00745; A61B 2017/00199; A61B 2217/005

(57) ABSTRACT

Provided herein are systems, methods, and apparatuses that include a vitrectomy tip having port features for improved severing of vitreous fibers in a vitrectomy procedure. The vitrectomy tip may include one or more filaments traversing a port formed in the vitrectomy tip. The one or more filaments separate the port into a plurality of openings. When the vitrectomy tip is ultrasonically vibrated, the filaments impact the vitreous fibers causing them to sever. In this way, the filaments serve as severing elements to separate the vitreous fibers from the eye, thereby providing or improving the ability of the vitrectomy tip to perform the vitrectomy procedure.

### TECHNICAL FIELD

The present disclosure is directed to systems, instruments, and methods for use in medical procedures, and, more particularly, to systems, instruments, and methods for vitrectomy procedures.

### BACKGROUND

Vitreo-retinal procedures are commonly performed within the posterior chamber of the human eye to treat many serious conditions of the posterior segment of the eye. In particular, vitreo-retinal procedures may treat conditions such as age-related macular degeneration (AMD), diabetic retinopathy and diabetic vitreous hemorrhage, macular hole, retinal detachment, epiretinal membrane, cytomegalovirus (CMV) retinitis, and many other ophthalmic conditions.

Such procedures frequently require the cutting and removal of portions of the vitreous humor from the posterior segment of the eye. The vitreous humor is comprised of microscopic fibers or strands within the posterior chamber. A surgeon performs vitreo-retinal procedures with a microscope and special lenses designed to provide a clear image of the posterior segment. Several tiny incisions just a millimeter or so in diameter are typically made on the sclera at the pars plana. In a vitrectomy procedure, the surgeon inserts microsurgical instruments through the incisions, including a vitrectomy probe or tip to cut and remove the strands of the vitreous body.

Examples of vitrectomy probes are disclosed, for example, in U.S. Patent Application No. 2014/0171997, U.S. Patent Application No. 2014/0364886, and U.S. Patent Application No. 2015/0173948, the disclosures of which are incorporated by reference herein.

In certain prior vitrectomy probes, the instrument includes an external tube with a port or hole in the tube, for example in the side of the tube. The instrument further includes an internal cutting tube within the external tube, the internal cutting tube having a cutting surface at a distal edge thereof. Suction is applied to draw the vitreous fibers into the port of the external tube, while the internal cutting tube reciprocates at high speed. As the internal cutting tube approaches and passes by the port, the action of the cutting edge of the internal cutting tube against the vitreous fibers cuts or breaks the fibers such that the vitreous fibers can be suctioned away and removed.

Certain ophthalmological instruments use ultrasound energy to deliver into the eye for the desired procedure. For example, in cataract surgery, ultrasonic energy is commonly used during phacoemulsification of the natural lens, serving to break the natural lens into fragments that can be aspirated away.

In ultrasonic vitrectomy probes, the instrument uses ultrasonic action to cut or break the vitreous fibers. In an example, the instrument includes a tube with a port, into which the vitreous fibers are drawn by suction. The high speed ultrasonic oscillation of the instrument causes the fibers to cut or break such that they can be suctioned away and removed.

The removal of vitreous fibers is a sensitive procedure which must be performed efficiently and without damage to the retina or other parts of the eye. Accordingly, it is desired to improve upon existing vitrectomy probes.

### SUMMARY

Improvements in ultrasonic vitrectomy instruments and associated systems and methods are disclosed herein.

According to one aspect, an ultrasonic vitrectomy instrument includes a handpiece including an ultrasonic horn operable to transmit ultrasonic vibrations and a vitrectomy tip attached to the handpiece. The vitrectomy tip is operable to vibrate in response to the ultrasonic vibrations transmitted by the ultrasonic horn. The vitrectomy tip may include a proximal end, a distal end, an elongated portion extending from the proximal end to the distal end, a port located at the distal end of the vitrectomy tip and a least one filament separating the portion into a plurality of openings.

Another aspect includes a system for an ultrasonic vitrectomy procedure including a console comprising a control system and a handpiece connected to the console. The control system of the console may be configured to control ultrasonic vibrations generated by the handpiece. The handpiece may include an ultrasonic horn operable to transmit ultrasonic vibrations, an electrical cable extending between the handpiece and the control system, an aspiration line extending between the handpiece and the console, and a vitrectomy tip attached to the handpiece and defining a channel in fluid communication with the aspiration line. The electrical cable may be configured to transmit control signals to the handpiece. The control signals may be operable to control the generation of the ultrasonic vibrations. The aspiration line may be configured to convey aspirated materials from the handpiece to the console. The vitrectomy tip may be configured to vibrate ultrasonically in response to the ultrasonic vibrations transmitted to the vitrectomy tip from the ultrasonic horn. The vitrectomy tip may include a proximal end, a distal end, an elongated portion extending from the proximal end and the distal end, a port formed in the elongated portion at the distal end of the vitrectomy tip, and at least one filament separating the port into a plurality of openings. The port may provide fluid communication between the channel and an exterior of the vitrectomy tip and may be configured to receive vitreous fibers.

Another aspect may include a method of performing a vitrectomy procedure including inserting a vitrectomy tip defining a channel extending therethrough and coupled to a handpiece into a posterior segment of an eye, ultrasonically vibrating the vitrectomy tip within the posterior segment while applying suction to the longitudinal tube of the vitrectomy tip, severing vitreous fibers with at least one filament, and aspirating the severed vitreous fibers from the posterior segment of the eye. The handpiece may include an ultrasonic horn configured to transmit ultrasonic vibrations to the vitrectomy tip. The vitrectomy tip may include a proximal end, a distal end, a longitudinal portion extending from the proximal end to the distal end, a port formed in the elongated portion at the distal end of the vitrectomy tip, and at least one filament separating the port into a plurality of openings. The port may provide fluid communication between the channel and an exterior of the vitrectomy tip. The at least one filament may separate the port into a plurality of openings.

Any of the different aspects may include one or more of the following features. The at least one filament may be a wire. The at least one filament may include at least two filaments separating the port into at least four openings. Each of the at least two filaments may be a wire. The handpiece may be adapted to transmit torsional ultrasonic vibrations to the vitrectomy tip. The torsional ultrasonic vibrations may cause oscillating movement of the vitrectomy tip in which the vitrectomy tip rotates back and forth between a first rotational direction and a second rotational direction. The torsional ultrasonic vibrations may cause the vitrectomy tip to rotate back and forth in angular rotations less than 360 degrees. The torsional ultrasonic vibrations may cause the vitrectomy tip to rotate back and forth in angular rotations less than 45 degrees. The handpiece may be adapted to transmit longitudinal ultrasonic vibrations to the vitrectomy tip. The handpiece may be adapted to transmit both torsional and longitudinal ultrasonic vibrations to the vitrectomy tip. The port located at the distal end of the vitrectomy tip may be formed at a distal tip of the elongated portion. The port may be formed in a sidewall of the elongated portion. The at least one filament may include two filaments separating the port into four openings.

It is to be understood that both the foregoing general description and the following detailed description are exemplary and explanatory in nature and are intended to provide an understanding of the present disclosure without limiting the scope of the present disclosure. In that regard, additional aspects, features, and advantages will be apparent to one skilled in the art from the accompanying drawings and the following detailed description.

### BRIEF DESCRIPTION OF THE DRAWINGS

The accompanying drawings illustrate implementations of the devices and methods disclosed herein and, together with the description, serve to explain the principles of the present disclosure.

FIG. 1 is a perspective view of an example control console and ultrasonic vitrectomy instrument.

FIG. 2 is a cross-sectional view of an example ultrasonic vitrectomy instrument that includes a handpiece and a vitrectomy tip attached to the handpiece.

FIG. 3 is a perspective view of an example vitrectomy tip.

FIG. 4 is a cross-sectional view of a portion of an example handpiece and the vitrectomy tip of FIG. 3 detached from the example handpiece.

FIG. 5 is a perspective view of the distal end of the vitrectomy tip of FIG. 3.

FIG. 6 is a perspective of example of a distal end of another example vitrectomy tip.

The accompanying drawings may be better understood by reference to the following detailed description.

### DETAILED DESCRIPTION

For the purposes of promoting an understanding of the principles described herein, reference will now be made to the implementations illustrated in the drawings, and specific language will be used to describe the same. It nevertheless will be understood that no limitation of the scope of the disclosure is intended. Any alterations and further modifications to the described devices, instruments, or methods, and any further application of the principles described herein, are fully contemplated as would normally occur to one skilled in the art to which the disclosure relates. In particular, it is fully contemplated that the features, components, and/or steps described with respect to one implementation may be combined with the features, components, and/or steps described with respect to other implementations of the present disclosure. For simplicity, in some instances the same reference numbers are used throughout the drawings to refer to the same or like parts.

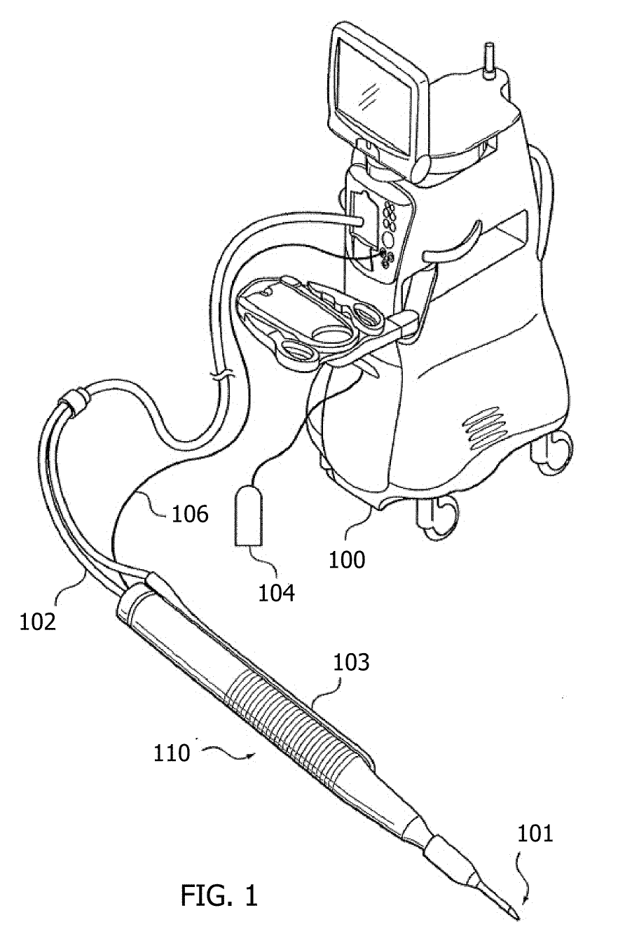

FIG. 1 is an example of a surgical console 100 that may be similar, for example, to that depicted in U.S. Pat. No. 8,579,929, the disclosure of which is incorporated herein by reference. The surgical console 100 may be, for example, the INFINITI® Vision Systems available from Alcon Laboratories, Inc. of Fort Worth, Tex. Console 100 is connected to an ultrasonic vitrectomy instrument 110 through aspiration line 102 and irrigation line 103. The fluid flows through lines 102 and 103 are controlled by the user, for example, via foot pedal 104. Power is supplied to the ultrasonic handpiece 110 through an electrical cable 106.

As shown in FIG. 1, the ultrasonic vitrectomy instrument 110 is attached to an ultrasonic phacoemulsification needle tip 101 for use in cataract surgery. The phacoemulsification needle tip 101 may be straight or bent or angled.

The ultrasonic vitrectomy instrument 110 may be similar, for example, to that depicted in U.S. Pat. No. 6,402,769 or U.S. Pat. No. 7,651,490, the disclosures of which are incorporated herein by reference. The ultrasonic vitrectomy instrument 110 includes a handpiece that may be, for example, the OZIL® Handpiece available from Alcon Laboratories, Inc. of Fort Worth, Tex. The ultrasonic vitrectomy instrument 110 may include an ultrasonic handpiece that can produce torsional ultrasonic vibrations, longitudinal ultrasonic vibrations, or both. The mode of operation (torsional or longitudinal) and the frequency of the vibrations may be selected by operator input to the surgical console 100.

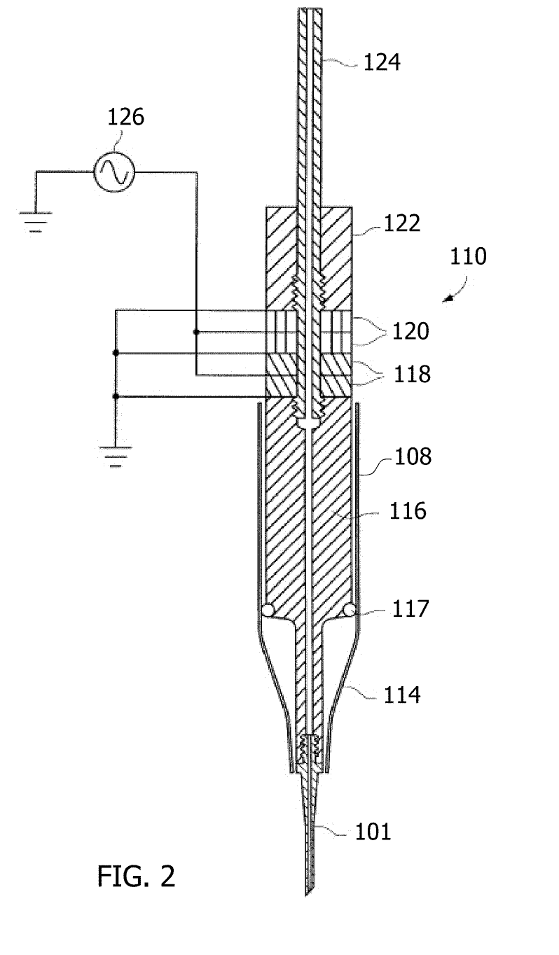

FIG. 2 is an example of an ultrasonic vitrectomy instrument 110 similar to that may be similar to that depicted in U.S. Pat. No. 6,402,769. As can be seen in FIG. 2, the ultrasonic vitrectomy instrument 110 includes a handpiece 108 and a tip 101 coupled to the handpiece 108. The handpiece 108 includes a handpiece shell 114, ultrasound horn 116, torsional ultrasound crystals 118, and longitudinal ultrasound crystals 120. The horn 116 is held within the shell 114 by an isolator 117. The crystals 118 and 120 are held within the shell 114 and in contact with the horn 116 by a back cylinder 122 and a bolt 124. The crystals 118 and 120 vibrate ultrasonically in response to a signal generated by an ultrasound generator 126. The crystals 118 are polarized to produce torsional motion. The crystals 120 are polarized to produce longitudinal motion. In an alternative, as illustrated in FIG. 6 of U.S. Pat. No. 6,402,769, the handpiece may have one or more crystals used to produce both longitudinal and torsional motion.

The signal generated by the ultrasound generator 126 may be controlled by an operator, e.g., a surgeon or other medical professional, using the control system of the surgical console 100. The signals from the surgical console 100 are transmitted through the electrical cable 106 to the crystals 118 and 120 of the handpiece 108.

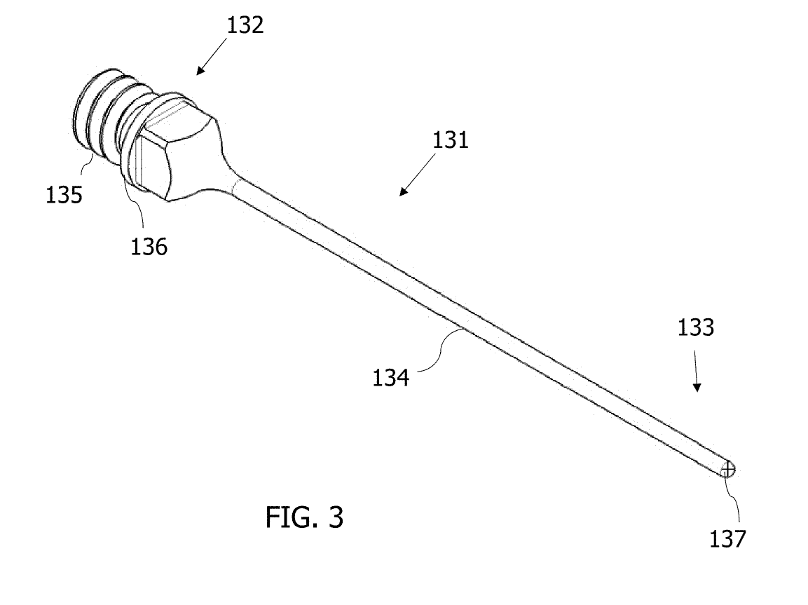

FIG. 3 is a perspective view of an example vitrectomy tip 131. The vitrectomy tip 131 may be attached to a handpiece, such as the handpiece 108. For example, the vitrectomy tip 131 may be attached to the handpiece 108 in place of vitrectomy tip 101. The vitrectomy tip 131 may be attached to the handpiece 108 in numerous ways, such as, for example, a threaded connection, snap fit, cam fit, interlocking lugs, or any other suitable connection. The vitrectomy tip 131 is connected to the handpiece 108 so that the ultrasonic vibrations produced by the crystals 118 or 120 are transmitted from the ultrasonic horn 116 to the vitrectomy tip 131.

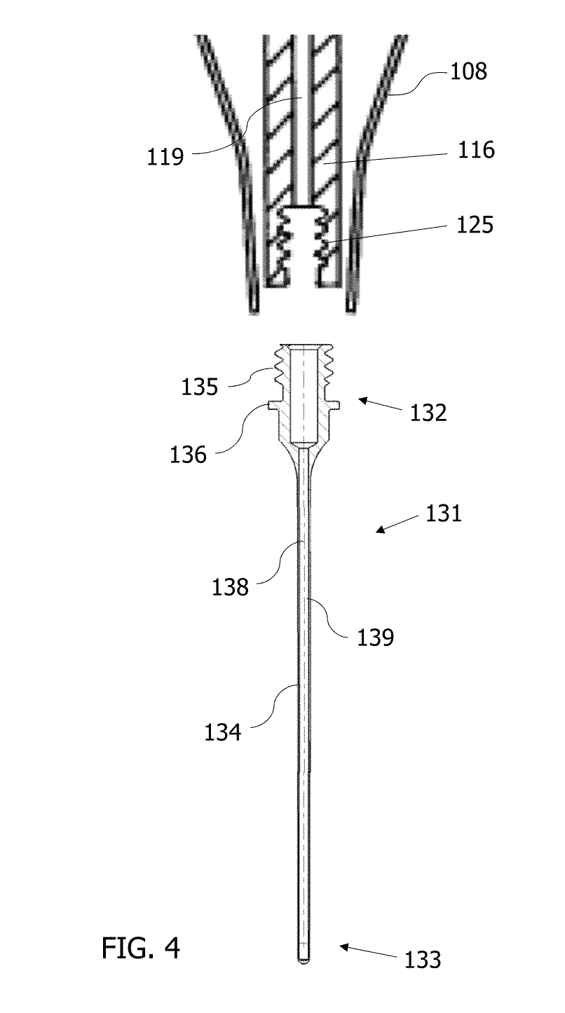

The vitrectomy tip 131 includes a proximal end 132, a distal end 133, and an elongated portion 134 extending from the proximal end 132 to the distal end 134. The vitrectomy tip 131 defines a channel 139, shown in FIG. 4, that terminates at a port 137 formed at the distal end 133 of the vitrectomy tip 131. Materials, such as fluid and tissues (e.g., vitreous fibers), may be aspirated from an eye by entering the port 137 and passing along the channel 139.

FIG. 4 shows a cross-sectional view of a distal portion of the handpiece 108 and the vitrectomy tip 131. The handpiece 108 includes a connection element, for example, a threaded element 125. The threaded element 125 may be, for example, an internally-threaded bore of the ultrasonic horn 116. The vitrectomy tip 131 also includes a corresponding connection element, for example, a threaded element 135. The threaded element 135 may be, for example, an externally-threaded part at the proximal end 132 of the vitrectomy tip 131. The vitrectomy tip 131 may further include a flange 136. The flange 136 may act as a stop to limit a distance into which the vitrectomy tip 131 may be received into the handpiece 108 (e.g., into the internally-threaded bore of the ultrasonic horn 116).

When connected to the handpiece 108, the channel 139 adjoins a channel 119 extending through the handpiece 108. The channel 139 and channel 119 form a passage through which material is aspirated through the ultrasonic vitrectomy instrument 110. The passage connects with an aspiration line, such as the aspiration line 102 shown in FIG. 1. The material aspirated through the ultrasonic vitrectomy instrument 110 is passed along into the aspiration line 102 and discarded. The channel 119 extending through the handpiece 108 may extend through the ultrasonic horn 116 and/or other components such that the channel 119 is in fluid communication with the aspiration line 102 The channel 119 and the aspiration line 102 may fluidly communicate with each other such that a fluid, e.g., a liquid or gas, passes feely therebetween. In this manner, the aspiration line 102 communicates a suction or vacuum from the surgical console 100 through the channel 119 of the handpiece 108, the channel 139 134, and to the port 137. A reduced pressure or vacuum source in the surgical console 100 draws or aspirates the vitreous fibers, as well as other materials, from the eye through the port 137, the channel 139, the channel 119, and the aspiration line 102. The aspirated materials may be collected in a collection device or container. The aspiration may be aided by a saline flushing solution or irrigant, such as BSS® or BSS PLUS® produced by Alcon Laboratories, Inc., located at 6201 South Freeway, Fort Worth, Tex. 76134, that is introduced into the surgical site through the irrigation line 103.

When the vitrectomy tip 131 is connected to the handpiece 108, ultrasonic vibrations produced by the crystals 120 or 118 are transmitted to the vitrectomy tip 131. The ultrasonic vibrations may be torsional vibrations, longitudinal vibrations, or a combination of torsional and longitudinal vibrations. The control, frequency, and extent of such vibrations may be effected as known, for example, from the OZIL® Handpiece mentioned above, or as shown and described in U.S. Pat. No. 6,402,769, U.S. Pat. No. 7,651,490, or U.S. Pat. No. 8,579,929, the disclosures of which are incorporated herein by reference. In an example, the handpiece 108 is capable of torsional and longitudinal vibrations, and the torsional vibration mode or the longitudinal vibration mode may be selected using the surgical console 100.

In torsional vibration mode, the handpiece 108 causes the vitrectomy tip 131 to rotate or twist about its longitudinal axis 138 at ultrasonic frequency. The torsional rotation rapidly occurs in opposing directions, e.g., clockwise then counterclockwise then clockwise again and so on, such that the motion is oscillatory about the longitudinal axis 138. Thus, the torsional ultrasonic vibrations cause oscillating movement of the vitrectomy tip 131 in which the vitrectomy tip 131 rotates back and forth between a first rotational direction and a second rotational direction. With each directional movement, the rotation may be less than a full revolution. Thus, the vitrectomy tip may rotate back and forth in angular rotations less than 360 degrees. For example, the handpiece 108 may transmit torsional ultrasonic vibrations to the vitrectomy tip 131 that causes the vitrectomy tip 131 to rotate back and forth in angular rotations less than 45 degrees. In some implementations, the ultrasonic rotation of the vitrectomy tip 131 may be 45 degrees, 40 degrees, 35 degrees, 30 degrees, or less than 30 degrees. Still further, the angular rotation of the vitrectomy tip 131 may be values between those listed or greater than or less than those listed. Other amounts of rotation may be selected depending on the desired application.

In longitudinal vibration mode, the handpiece 108 causes the vitrectomy tip 131 to move forward and backward along the longitudinal axis 138 at ultrasonic frequency. As with torsional vibration mode, the frequency and extent of fore and aft displacement of the longitudinal vibrations may be selected depending on the desired application and controlled using the control system of the surgical console 100, for example. In other instances, a controller for controlling the longitudinal or torsional vibrations may be separate from the surgical console 100.

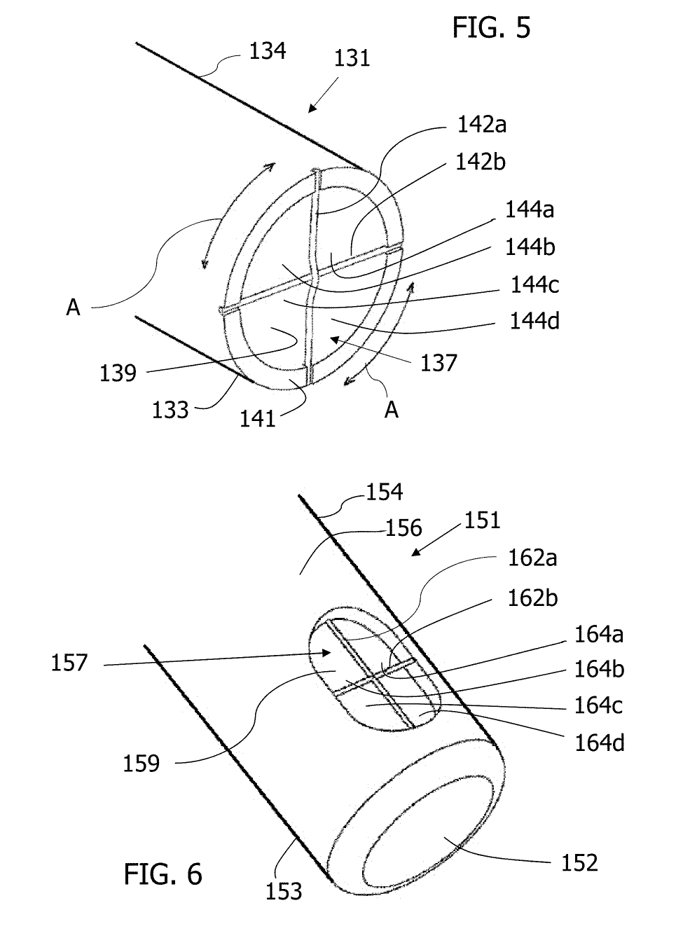

FIG. 5 is a perspective view of the distal end 133 of the vitrectomy tip 131 of FIG. 3. As shown in FIG. 5, the channel 139 extends to the port 137, providing fluid communication between the channel 139 and an exterior of the vitrectomy tip 31. As shown in FIG. 5, the port 137 is formed in a distal end face 141 of the vitrectomy tip 131. In some instances, the distal end face 141 may be perpendicular to the longitudinal axis 138. In other instances, the distal end face 141 may be beveled and form an oblique angle with the longitudinal axis 138.

As shown in FIG. 5, the vitrectomy tip 131 includes a first filament 142a and a second filament 142b traversing the port 137. In the illustrated example, the first and second filaments 142a and 142b are 90° offset from each other and cross over each other to divide. As a result the port 137 is divided into a plurality of quadrants or openings 144a, 144b, 144c, 144d. In some implementations, each of the filaments 142a, 142b may be a wire. The filaments 142a, 142b may be connected to the elongated portion 134 in any desired or suitable manner. For example, the filaments 142a and 142b may be connected to the elongated portion 134 by welding, by integral formation with the elongated portion 134, by removal of material, or in any other applicable manner.

In other implementations, the filaments 142a and 142b may be arranged other than perpendicular to each other. That is, filaments 142a and 142b may cross each other to define oblique angles. In still other implementations, the vitrectomy tip 131 may include a single filament extending across the port 137 and dividing the port 137 into two separate openings. In some instances, the single filament may divide the port 137 into two equal parts. In still other implementations, the single filament may divide the port 137 into unequal parts.

FIG. 6 shows a perspective view of a distal end 153 of another example vitrectomy tip 151. The vitrectomy tip 151 may be similar in all respects to the vitrectomy tip 131 except that the distal tip 152 of the vitrectomy tip 151 is closed, and a port 157 is formed in a sidewall 156 of an elongated portion 154 of the vitrectomy tip 151. The port 157 is located proximate to the distal tip 152 and connects to channel 159 that extends longitudinally through the vitrectomy tip 151. The port 157 provides fluid communication between the channel 159 and the exterior of the vitrectomy tip 151. The vitrectomy tip 151 may be connected to and used with a handpiece and surgical console in a similar manner as the vitrectomy tip 131.

As shown in FIG. 6, the vitrectomy tip 151 includes filaments 162a and 162b that traverse the port 157. The filaments 162a and 162b may be arranged perpendicular to each other and cross each other so as to divide the port 157 into a plurality of openings 164a, 164b, 164c, 164d. In some instances, each of the filaments 162a, 162b may be a wire. The filaments 162a, 162b may be connected to the elongated portion 154 in any desired or suitable manner. For example, the filaments 162a and 162b may be connected to the elongated portion 154 by welding, by integral formation with the elongated portion 154, by removal of material, or in any other applicable manner.

In other implementations, the filaments 162a and 162b may be arranged other than perpendicular to each other. That is, filaments 162a and 162b may cross each other to define oblique angles. In still other implementations, the vitrectomy tip 131 may include a single filament extending across the port 157 and dividing the port 157 into two separate openings. In some instances, the single filament may divide the port 157 into two equally sized portions. In still other implementations, the single filament may divide the port 157 into unequally sized portions.

The filaments 142a, 142b, 162a, and 162b facilitate the removal of vitreous fibers. The vitreous fibers are drawn into the openings 144a, 144b, 144c, 144d, 164a, 164b, 164c, 164d by suction applied to the respective channels 139 and 1159, and the movement of the filaments by ultrasonic vibration against the vitreous fibers severs the fibers (by breaking or cutting them) to allow the vitreous fibers to be suctioned away and out of the eye.

While FIGS. 5 and 6 show two filaments dividing the respective ports 137 and 157, the scope is not so limited. Rather, in other implementations, more than two filaments may be used to further divide the ports into more than four openings. Thus, in other implementations, three, four, or more filaments may be used to divide the port in to a plurality of openings.

As an example, when the vitrectomy tip 131 is operated in torsional ultrasonic mode, the vitrectomy tip 131 rotationally oscillates back and forth as indicated by the arrows A in FIG. 5. In the absence of the filaments 142a, 142b, the port 137 may have limited action against the vitreous fibers suctioned into the port 137, with limited ability to sever the vitreous fibers. In the center of the port 137 in particular, a lowest pressure differential exists and no structure is present to cause cutting of the vitreous fibers. With the filaments 142a, 142b in place, the rotation of the vitrectomy tip 131 causes the filaments 142a, 142b to impact against and sever the vitreous fibers, e.g., by breaking or cutting the vitreous fibers. The filaments 162a and 163b operate in a similar manner. The filaments 142a, 14b, 162a, 162b provide or create additional high speed contact surfaces or high pressure areas for action against the vitreous fibers.

In an example method of performing a vitrectomy procedure, the operator attaches a vitrectomy tip, such as vitrectomy tip 131 or 151, to a handpiece, such as handpiece 108, which is in turn connected to a surgical console, such as surgical console 100. The user, such as a physician or other medical professional, inserts a vitrectomy tip into an eye such as by access procedures known in the art. For example, the vitrectomy tip may be inserted through the pars plana into the posterior segment of the eye with or without the use of a trocar cannula. Suction is applied by the surgical console through an aspiration line, such as aspiration line 102, and an internal channel, such as internal channel 139 or 159, of the vitrectomy tip. By control of the surgical console, the handpiece is operated in the desired mode, for example, torsional or longitudinal ultrasonic mode, causing ultrasonic vibrations at the vitrectomy tip. By ultrasonically vibrating the vitrectomy tip within the posterior segment while applying suction to the elongated portion of the vitrectomy tip (e.g., elongated portion 134), the ultrasonic vibration of filament(s) of the vitrectomy tip, such as the filaments 142a, 142b, 162a, or 162b, against the vitreous fibers severs the vitreous fibers. The severed fibers then may be aspirated through the handpiece and the aspiration line 102 and out of the posterior segment of the eye.

Thus, as described above, provided herein, among other systems, methods, and apparatuses, are vitrectomy tips having port features for improved or enhanced severing of vitreous fibers in a vitrectomy procedure. The port features may include one or more filaments traversing the port of the vitrectomy tip, thereby separating the port into a plurality of openings. When the tip is ultrasonically vibrated, the filaments impact the vitreous fibers causing the filaments to sever. The action of the filaments against the vitreous fibers provides a severing action for separating the vitreous fibers from the eye for removal. In this way, the filaments serve as severing elements to separate the vitreous fibers from the eye, thereby providing or improving the ability of the vitrectomy tip to perform the vitrectomy procedure.

A vitrectomy tip in accordance with implementations of the disclosure may be made of any suitable material. For example, the vitrectomy tip may be formed from a metal, such as, for example, a stainless steel or a titanium alloy. The vitrectomy tip may be sized and shaped and have associated dimensions for use in a vitrectomy procedure. For example, the elongated portion may have an outer diameter sized for a 23 gauge, 25 gauge, or 27 gauge procedure. In one example, the outer diameter of the elongated portion may be approximately 0.0255 inches.

Persons of ordinary skill in the art will appreciate that the implementations encompassed by the present disclosure are not limited to the particular exemplary implementations described above. In that regard, although illustrative implementations have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ultrasonic vitrectomy instrument comprising:
a handpiece comprising an ultrasonic horn operable to transmit ultrasonic vibrations; and
a vitrectomy tip attached to the handpiece and operable to vibrate in response to the ultrasonic vibrations transmitted by the ultrasonic horn, the vitrectomy tip comprising:
a proximal end;
a distal end;
an elongated portion extending from the proximal end to the distal end;
a port located at the distal end of the vitrectomy tip; and
at least one filament separating the port into a plurality of openings.

2. The ultrasonic vitrectomy instrument according to claim 1, wherein the at least one filament is a wire.

3. The ultrasonic vitrectomy instrument according to claim 1, wherein the at least one filament comprises at least two filaments separating the port into at least four openings.

4. The ultrasonic vitrectomy instrument according to claim 3, wherein each of the at least two filaments is a wire.

5. The ultrasonic vitrectomy instrument according to claim 1, wherein the handpiece is adapted to transmit torsional ultrasonic vibrations to the vitrectomy tip.

6. The ultrasonic vitrectomy instrument according to claim 5, wherein the torsional ultrasonic vibrations cause oscillating movement of the vitrectomy tip in which the vitrectomy tip rotates back and forth between a first rotational direction and a second rotational direction.

7. The ultrasonic vitrectomy instrument according to claim 6, wherein the torsional ultrasonic vibrations cause the vitrectomy tip to rotate back and forth in angular rotations less than 360 degrees.

8. The ultrasonic vitrectomy instrument according to claim 6, wherein the torsional ultrasonic vibrations cause the vitrectomy tip to rotate back and forth in angular rotations less than 45 degrees.

9. The ultrasonic vitrectomy instrument according to claim 1, wherein the handpiece is adapted to transmit longitudinal ultrasonic vibrations to the vitrectomy tip.

10. The ultrasonic vitrectomy instrument according to claim 1, wherein the handpiece is adapted to transmit both torsional and longitudinal ultrasonic vibrations to the vitrectomy tip.

11. The ultrasonic vitrectomy instrument according to claim 1, wherein the port located at the distal end of the vitrectomy tip is formed at a distal tip of the elongated portion.

12. The ultrasonic vitrectomy instrument according to claim 1, wherein the port is formed in a sidewall of the elongated portion.

13. A system for an ultrasonic vitrectomy procedure comprising:
a console comprising a control system;
a handpiece connected to the console, the control system of the console configured to control ultrasonic vibrations generated by the handpiece, the handpiece comprising:
an ultrasonic horn operable to transmit ultrasonic vibrations;
an electrical cable extending between the handpiece and the control system, the electrical cable configured to transmit control signals to the handpiece, the control signals operable to control the generation of the ultrasonic vibrations;
an aspiration line extending between the handpiece and the console, the aspiration line configured to convey aspirated materials from the handpiece to the console; and
a vitrectomy tip attached to the handpiece and defining a channel in fluid communication with the aspiration line, the vitrectomy tip configured to vibrate ultrasonically in response to the ultrasonic vibrations transmitted to the vitrectomy tip from the ultrasonic horn, the vitrectomy tip comprising:
a proximal end;
a distal end;
a elongated portion extending from the proximal end to the distal end;
a port formed in the elongated portion at the distal end of the vitrectomy tip, the port providing fluid communication between the channel and an exterior of the vitrectomy tip and configured to receive vitreous fibers; and
at least one filament separating the port into a plurality of openings.

14. A method of performing a vitrectomy procedure comprising:
inserting a vitrectomy tip defining a channel extending therethrough and coupled to a handpiece into a posterior segment of an eye, the handpiece comprising an ultrasonic horn configured to transmit ultrasonic vibrations to the vitrectomy tip, the vitrectomy tip comprising:
a proximal end;
a distal end;
a longitudinal portion extending from the proximal end to the distal end;
a port formed in the elongated portion at the distal end of the vitrectomy tip, the port providing fluid communication between the channel and an exterior of the vitrectomy tip; and
at least one filament separating the port into a plurality of openings;
ultrasonically vibrating the vitrectomy tip within the posterior segment while applying suction to the longitudinal tube of the vitrectomy tip;
severing vitreous fibers with the at least one filament; and
aspirating the severed vitreous fibers from the posterior segment of the eye.

15. The method according to claim 14, wherein the at least one filament is a wire.

16. The method according to claim 14, wherein the at least one filament comprises two filaments separating the port into four openings.

17. The method according to claim 16, wherein each of the two filaments is a wire.

18. The method according to claim 14, wherein the handpiece transmits torsional ultrasonic vibrations to the vitrectomy tip.

19. The method according to claim 18, wherein the torsional ultrasonic vibrations cause oscillating movement of the vitrectomy tip in which the vitrectomy tip rotates back and forth between a first rotational direction and a second rotational direction.

20. The method according to claim 18, wherein the handpiece transmits both torsional and longitudinal ultrasonic vibrations to the vitrectomy tip.
